(19) United States
(12) Patent Application Publication
(10) Pub. No.: US 2006/0025823 A1
(43) Pub. Date: Feb. 2, 2006

(54) Method and apparatus for facilitating opening a sealed medical electrode package

(75) Inventor: Eric Jonsen, Seattle, WA (US)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics, N.V., Eindhoven (NL)

(21) Appl. No.: 10/531,974

(22) PCT Filed: Oct. 13, 2003
(86) PCT No.: PCT/IB03/04500

§ 371 Date: Apr. 20, 2005

Related U.S. Application Data
(60) Provisional application No. 60/420,765, filed on Oct. 23, 2002.

(51) Int. Cl. A61N 1/39

(52) U.S. Cl. 607/5

(57) ABSTRACT

An apparatus for storing one or more electrodes for an automatic external defibrillator includes inter alia a cavity and a lid. The cavity accepts the one or more electrodes and includes at least one vertical surface. A top lateral edge surface of the cavity meets the at least one vertical surface and has a first end with at least one hole. At least one ridge extends horizontally out from the at least vertical surface of the cavity. The lid is detachably attached to the cavity and includes a bottom surface that faces the top lateral edge surface of the cavity, as well as at least one protrusion that extends into the hole in the top lateral edge surface when the lid is attached to the cavity. The hole and the protrusion form a hinge structure at a hinged end of the lid and cavity. A handle is rotatably mounted to the lid opposite the hinged end. The handle includes a camming surface, which engages the ridge to create separation between the lid and the cavity as the handle is rotated from its closed position to its open position. A seal is fixed to the bottom surface and is also removably fixed to the top lateral edge surface of the cavity, thereby forming a sealed enclosure with the cavity in which the electrodes are disposed when the lid is attached to the cavity. As the handle is rotated from its closed position to its open position the seal is simultaneously peeled from the top lateral edge surface of the cavity.

### FIELD OF THE INVENTION

The present invention is directed to automatic external defibrillators, and more particularly to a method and apparatus for packaging and storing electrodes used by an automatic external defibrillator.

### BACKGROUND

As Automatic External Defibrillators (AEDs) become more widely available, the diversity of the user population also increases. Therefore, electrode-packaging designs must take into account a broader range of physical strength and dexterity. In the past, the primary users of AEDs were highly trained emergency medical personnel. These professionals were generally more physically fit than the average layperson due to the demanding nature of their work. Now, the user population includes people (e.g., senior citizens) who may not have the required strength or dexterity to open a sealed medical electrode package although they are otherwise able to use an AED. Inability to open the electrode package constitutes an insurmountable barrier to using an AED device, especially during high stress situations in which these devices are typically employed. As such, the market for these devices remains limited. Moreover, the packaging options are also limited for similar reasons.

The present invention is therefore directed to the problem of developing a method and apparatus for facilitating the opening of a sealed medical electrode package that will allow those with limited physical strength and dexterity to open these packages.

### SUMMARY OF THE INVENTION

The present invention solves these and other problems by providing inter alia, a handle to provide a mechanical advantage when opening a sealed medical electrode package. Another possible embodiment of the present invention includes a blade mounted on a track that is integral to the package lid.

Still another possible embodiment of the present invention includes a wire or string embedded in the seal material that when pulled cuts through the seal and opens the package similar to opening a roll of round hard candies.

Yet another possible embodiment of the present invention includes a sharp point disposed on a rigid lid or frame, which punctures the seal material to initiate a tear. Yet another possible embodiment of the present invention includes a feature that functions like the tab used to open a can of pop or roasted nuts. This embodiment would be appropriate for a roll seal type lid.

Providing mechanical assistance in opening the electrode package significantly broadens the available market for AEDs, as well as increasing the packaging implementation options.

### BRIEF DESCRIPTION OF THE DRAWINGS

FIG. 1 depicts a top view of an exemplary embodiment of a cartridge assembly for storing and transporting electrodes for an automatic external defibrillator according to one aspect of the present invention.

FIG. 2 depicts a top view of a lower half of the embodiment of FIG. 1 according to another aspect of the present invention.

FIG. 3 depicts a side view of the embodiment of FIG. 1 in a closed position according to another aspect of the present invention.

FIGS. 4-6 depict in sequential side views the act of removing the lid from the embodiment of FIG. 1 according to another aspect of the present invention.

### DETAILED DESCRIPTION

It is worthy to note that any reference herein to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

According to one aspect of the present invention, an apparatus for storing one or more electrodes for an automatic external defibrillator includes a cavity and a lid. In some of the embodiments herein, a one-time seal is formed between the cavity and the lid.

The sealed cavity is for accepting the one or more electrodes. Preformed mountings can be disposed in the cavity to simplify the packing of the electrodes. One or more vertical sides form the sealed cavity. The cavity may be rectangular, circular or any other geometric shape in cross section; hence the number of vertical sides can be one, two, three, four or more. For example, in the case of a cylindrical structure, there is essentially only one vertical side, but in the case of a box structure, there are four vertical sides.

A top lateral edge surface, which extends perpendicularly relative to the vertical side(s), meets the vertical side(s) to completely enclose the cavity. A single use seal attaches to the top lateral edge surface, thereby forming the sealed cavity.

At least one ridge extends laterally out from at least one of the vertical sides of the cavity. A second ridge could be disposed on the opposite side of the cavity from the first ridge, or in the case of a cylindrical structure, the second ridge could be disposed approximately 180 degrees apart from the first ridge.

A hinge structure is formed at one end of the cavity. The hinge structure includes one or more holes in the cavity lip or lid and matching protrusions on the lid or lip of the cavity, respectively. This enables the lid to be detachably attached to the cavity by pivoting the lid about the hinge structure.

The single use seal is fixed to a bottom surface of the lid and is removably fixed to the top lateral edge surface of the cavity thereby forming a sealed enclosure in the cavity, in which the electrodes are disposed when the lid is attached to the cavity.

An axle is disposed at an opposite end from the hinge structure of the lid. The axle can be a single rod or two pins that attach to the lid.

A handle is mounted to the lid via the axle (or the two pins). The handle is rotatable about the axle (or two pins). The handle includes a camming surface that engages the one or more ridges as the handle is rotated about the axle to create separation between the lid and the cavity and to simultaneously peel the single use seal from the top lateral edge surface of the cavity as the handle is rotated from a closed position to an open position.

According to another aspect of the present invention, an apparatus for storing one or more electrodes for an automatic external defibrillator includes a container, a hinge structure and a handle.

The container includes a top portion, a bottom portion, a cavity for accepting the one or more electrodes. A single use seal is disposed between the top portion and the bottom portion. At least one lateral ridge extends outward from the container.

The hinge structure is disposed at a first end of the container. Any hinge structure will suffice for this purpose.

A handle is rotatably mounted to the container at an end opposite the first end. The handle includes a camming surface to contact the at least one lateral ridge. The camming surface engages the ridge as the handle is rotated from a closed position to an open position to create separation between the top portion and the bottom portion and to simultaneously remove or open the single use seal.

According to yet another aspect of the present invention, an apparatus for storing one or more electrodes for an automatic external defibrillator includes a container and a mechanically advantaged actuator.

The container includes a top portion, a bottom portion, a cavity for accepting the one or more electrodes. A single use seal is disposed between the top portion and the bottom portion. At least one lateral ridge extends outward from the container.

The mechanically advantaged actuator creates separation between the top portion and the bottom portion as the actuator is manipulated and to simultaneously removes the single use seal. An example of the mechanically advantaged actuator includes a handle with a cam providing a mechanical advantage to a person opening the sealed container. Other examples include a blade mounted on a track that is integral to the sealed container; a wire or string embedded in the seal material that when pulled cuts through the seal and opens the container; a sharp point disposed on a rigid lid or frame, which punctures the seal material to initiate a tear; or a feature that functions like the tab used to open a can of pop or roasted nuts, which would be appropriate for a roll seal type lid.

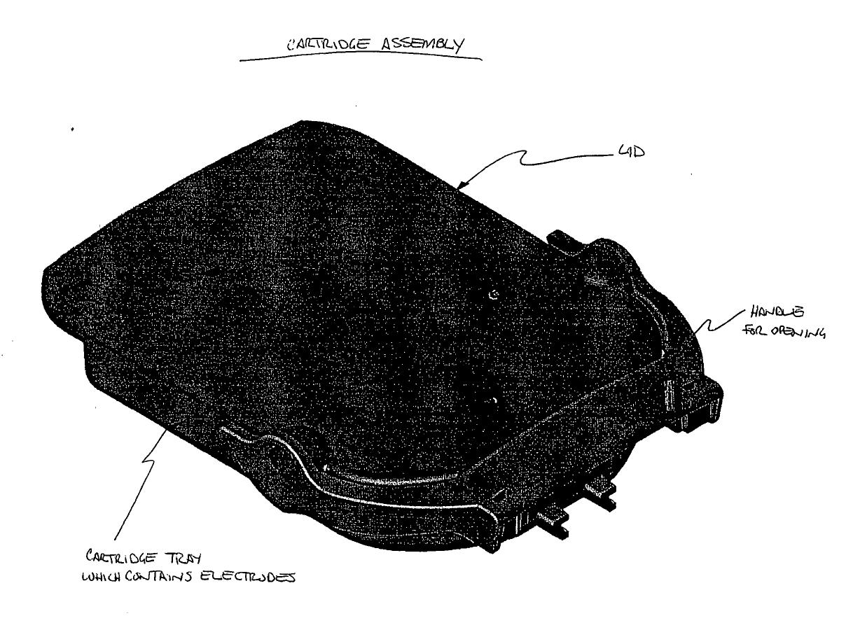

Turning to FIG. 1, shown therein is a top view of an exemplary embodiment 10 of a cartridge assembly for storing and transporting electrodes for an automatic external defibrillator according to one aspect of the present invention. This embodiment 10 includes a lid 11 and a tray 12 along with a handle 13 for removing the lid 11 from the tray 12. The electrodes are stored in the tray 12. The handle 13 provides the user a mechanical advantage, thereby reducing to a negligible level the physical strength and manual dexterity needed to open the sealed medical electrode package.

The container formed by the tray and lid may be composed of a rigid or semi-rigid material. Typically, this material should be sufficient to maintain a moist environment for several years for the electrodes. As the electrodes dry out, the impedance of the electrodes increases, thereby affecting the amount of current output at the point of application of the electrodes and making the electrodes and the AED less effective. Hence, the material should be such to maintain a water barrier, such as several millimeters thick aluminum or similar material.

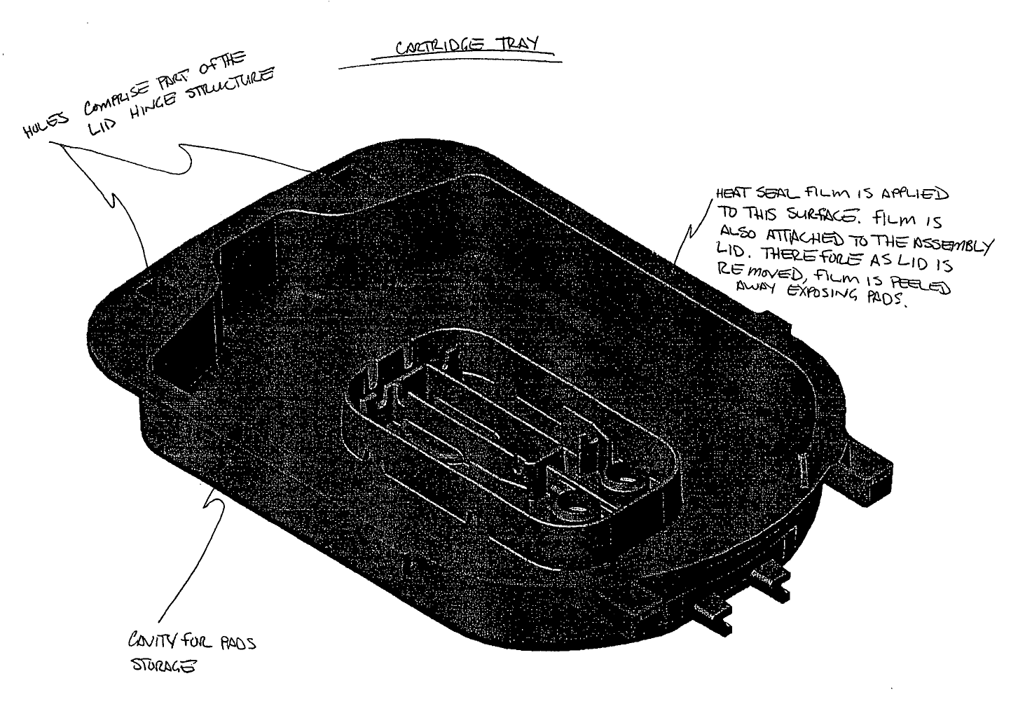

Turning to FIG. 2, shown therein is a top view of the cartridge tray 12, which stores the electrodes. A cavity 21 provides space for storing the pads and electrodes. A formed holding space 22 is provided in the center for accepting the electrodes and enabling the user to package the electrodes in a neat and orderly fashion. Two holes 23a-b form part of the lid hinge structure. A heat seal film (not shown) is applied to the surface 24 that meets with the lid 11. Similarly, a heat seal film (also not shown) is attached to the lid assembly 11. As the lid is removed from the cartridge 12, the heat seal film is peeled away thereby exposing the pads.

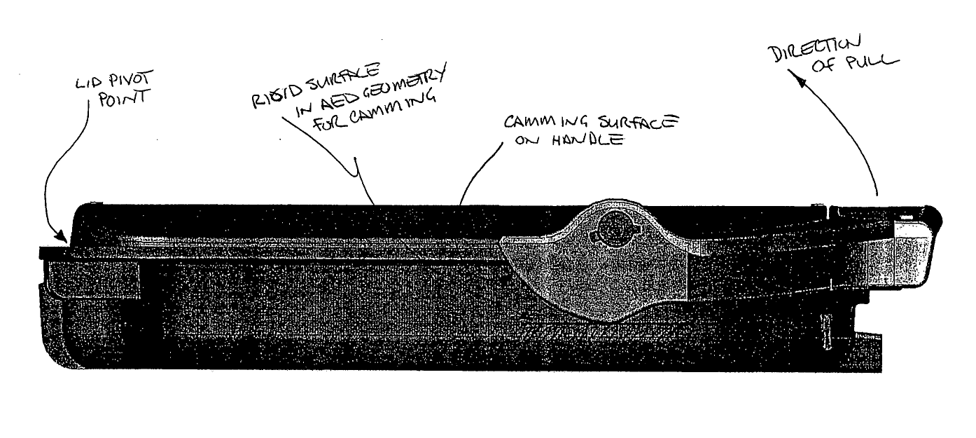

Turning to FIG. 3 shown therein is a side view of the cartridge assembly 10 with the lid 11 in a closed position. A pivot point 31 enables the opening of the lid 11 from one end. The side portion 32 has a rigid surface 33 along which a cam 34 on the handle 13 transfers the force of opening to separate the lid 11 from the cartridge 12. An axle 35 mounted in the side 32 allows the handle 13 to rotate about the axle 35 during motion. The handle 13 is pulled upwards and towards the pivot point 31. A similar construction exists on a side opposite the side shown in FIG. 3, thereby making a symmetric handle design and operation.

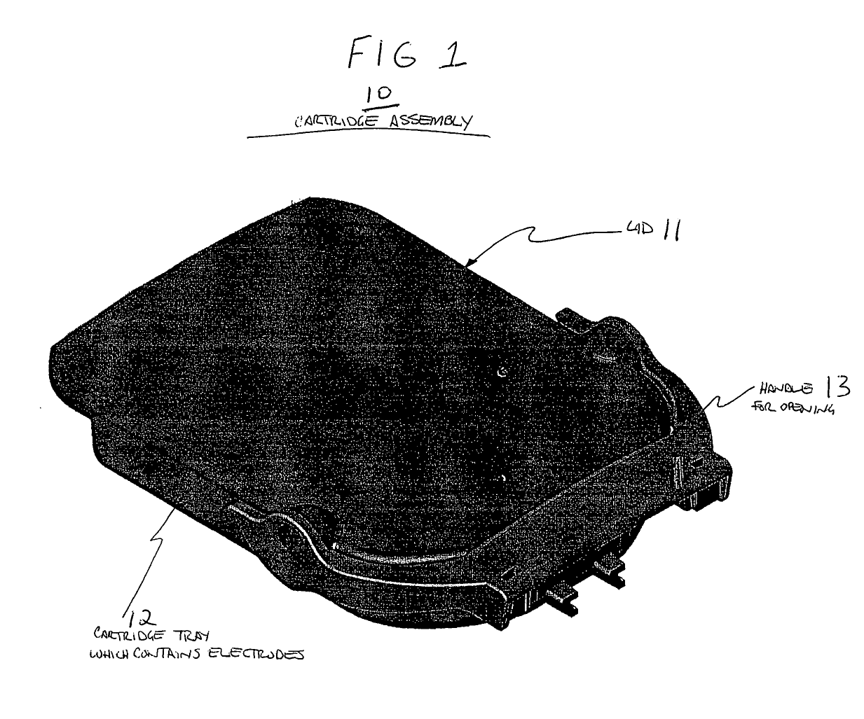

Turning to FIG. 4, shown therein is the cartridge assembly 10 as the handle 13 has been moved from its fully closed position to a partially open position. As can be seen in FIG. 4, the cam 34 has rotated along the rigid surface 33 as the handle 13 has been pulled upwards and in the direction of the pivot point 31. A point on the surface of the cam that is in contact with the rigid surface 33 is about ⅓ to ½ of the length of the surface of the cam 33 that contacts the rigid surface 33 during rotation of the cam 34. The heat seal film 41 is exposed and an initial peel of the heat seal film 41 has begun. The film 41 adheres to the lid 11 and peels away from the surface 24 of the cartridge assembly as the lid 11 is removed from the cartridge assembly 12. The axle 35 has been rotated about fifteen to thirty degrees.

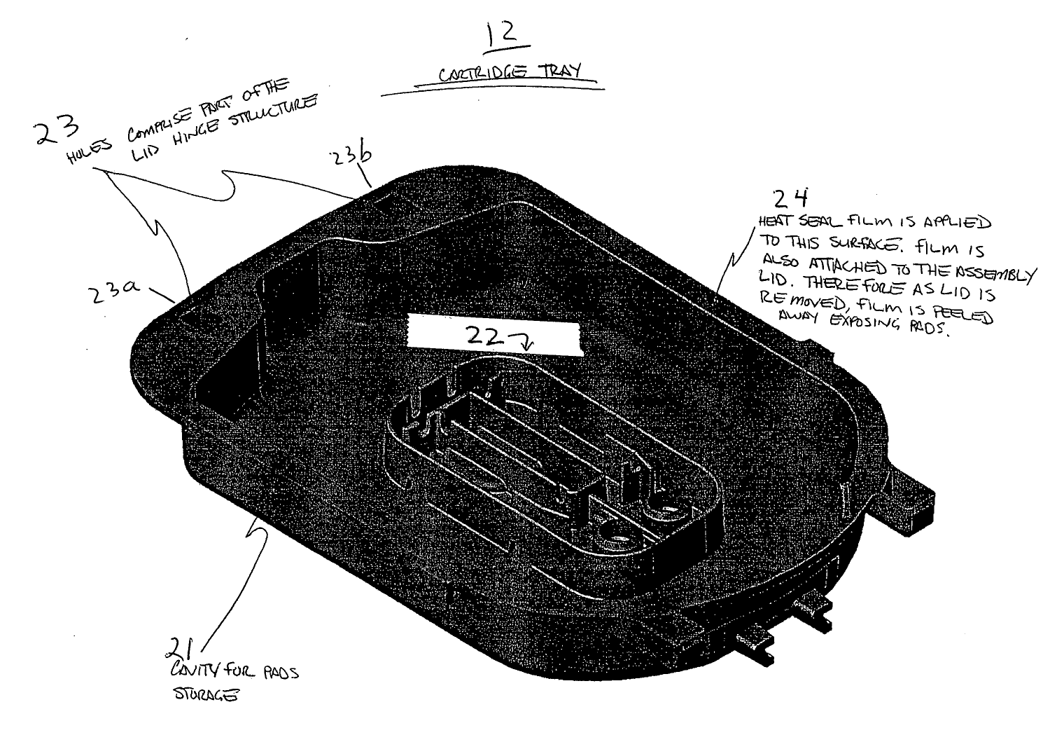

Turning to FIG. 5, shown therein is the cartridge assembly 10 as the handle 13 has been moved from its position in FIG. 4 to a more open position. As can be seen in FIG. 5, the cam 34 has rotated along the rigid surface 33 to a last point on the surface of the cam that slides along the rigid surface 33 as the handle 13 has been pulled upwards and in the direction of the pivot point 31. The heat seal film 41 is now more exposed and further peeling of the heat seal film 41 has occurred. In FIG. 5, the axle 35 has been rotated about ninety degrees. At this point, as the handle is pulled the film 41 peels away from surface 24. By now, the effort required to open the cartridge assembly is minimal, due only to that required to separate the film 41 from surface 24. Most of the force required to open the assembly 10 is due to the initial separation of the lid 11 from the cartridge 12. This is reduced to a negligible level by the handle and the cam.

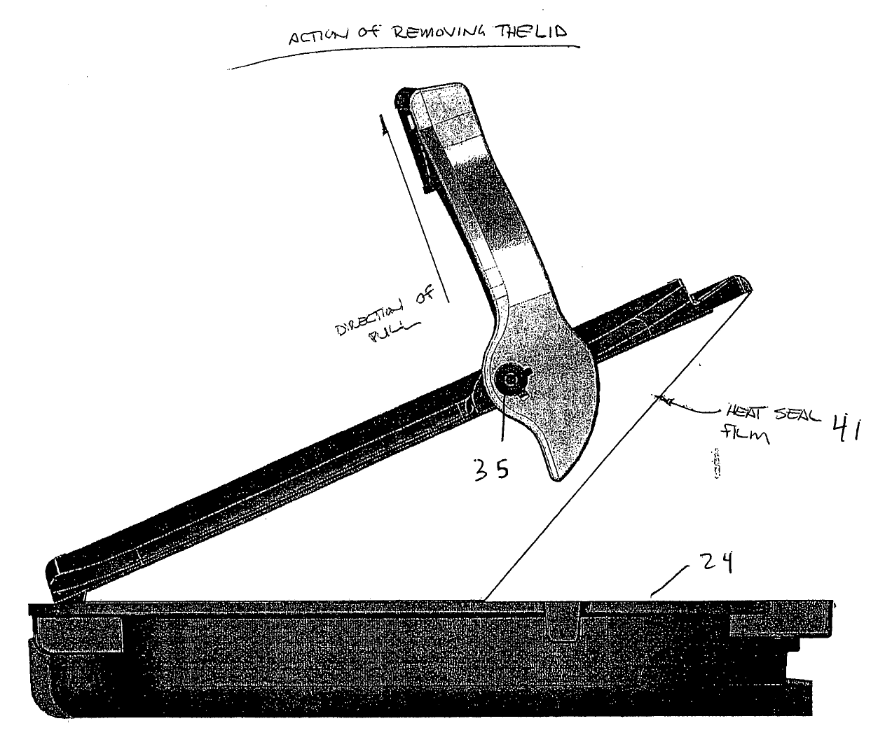

Turning to FIG. 6, shown therein is the cartridge assembly 10 as the handle 13 has been moved from its position in FIG. 5 to an almost completely open position. As can be seen in FIG. 6, the cam 34 is no longer in contact with the rigid surface 33 as the handle 13 has been pulled upwards and in the direction of the pivot point 31. The heat seal film 41 is now even more exposed and further peeling of the heat seal film 41 has occurred. In FIG. 6, the axle 35 has not been rotated any more than that shown in FIG. 5. Further rotation is not necessary as the handle is now being used to simply pull the heat seal film away from the surface 24.

Thus, the present invention reduces the physical strength and manual dexterity required to open a sealed medical electrode package. The seal can now be designed without compromising the strength of the seal or other physical characteristics otherwise necessary to facilitate human factors. Furthermore, a significantly wider range of sealing methods is now possible.

In an alternative embodiment, the heat seal film is separate from the cover. Thus, upon removing the cover, one must separately remove the heat seal film by pulling on a pull-tab, for example. Other standard techniques for removing the heat seal film can also be used.

Although various embodiments are specifically illustrated and described herein, it will be appreciated that modifications and variations of the invention are covered by the above teachings and are within the purview of the appended claims without departing from the spirit and intended scope of the invention. For example, the description above relates to a handle and a cam providing a mechanical advantage to a person opening the sealed medical electrode package; however, other possible implementations can also be used, such as: a blade mounted on a track that is integral to the package lid; a wire or string embedded in the seal material that when pulled cuts through the seal and opens the package; a sharp point disposed on a rigid lid or frame, which punctures the seal material to initiate a tear; or a feature that functions like the tab used to open a can of pop or roasted nuts, which would be appropriate for a roll seal type lid. Furthermore, these examples should not be interpreted to limit the modifications and variations of the invention covered by the claims but are merely illustrative of possible variations.

What is claimed is:

1. An apparatus for storing one or more electrodes for an automatic external defibrillator comprising:
a cavity for accepting the one or more electrodes, said cavity including:
at least one vertical surface;
a top lateral edge surface meeting the at least one vertical surface and having a first end with at least one hole; and
at least one ridge extending horizontally out from the at least vertical surface of the cavity; and
a lid detachably attached to the cavity, said lid including:
a bottom surface that faces the top lateral edge surface of the cavity;
at least one protrusion for extending into said at least one hole in the top lateral edge surface when the lid is attached to the cavity, wherein said at least one hole and said at least one protrusion form a hinge structure at a hinged end of the lid and cavity; and
a handle rotatably mounted to the lid opposite the hinged end, said handle including a camming surface, wherein said camming surface engages said at least one ridge to create separation between the lid and the cavity as the handle is rotated from a closed position to an open position.

2. The apparatus according to claim 1, further comprising a seal fixed to the bottom surface and removably fixed to the top lateral edge surface of the cavity forming a sealed enclosure with the cavity in which the electrodes are disposed when the lid is attached to the cavity.

3. The apparatus according to claim 2, wherein as the handle is rotated from the closed position to the open position the seal is simultaneously peeled from the top lateral edge surface of the cavity.

4. The apparatus according to claim 1, wherein the cavity includes a mounting bracket to which the electrodes are attached when stored.

5. The apparatus according to claim 1, wherein the lid and the cavity are made of a rigid material.

6. The apparatus according to claim 1, wherein the lid and the cavity are mode of a semi-rigid material.

7. The apparatus according to claim 1, further comprising one or more wires to couple the electrodes to an automatic external defibrillator when the electrodes are stored in the apparatus, said wires being coupled to the electrodes at the mounting bracket.

8. An apparatus for storing one or more electrodes for an automatic external defibrillator comprising:
a container including a top portion, a bottom portion, a cavity for accepting the one or more electrodes, at least one lateral ridge extending horizontally outward from the container, and a hinge disposed at a first end of the container; and
a handle rotatably mounted to the clam shell container at an end opposite the first end, said handle including a camming surface to contact the at least one lateral ridge, wherein said camming surface engages said at least one ridge as the handle is rotated from a closed position to an open position to create separation between the top portion and the bottom portion.

9. The apparatus according to claim 8, further comprising a single use seal disposed between the top portion and the bottom portion.

10. The apparatus according to claim 9, wherein as the handle is rotated from the closed position to the open position to create separation between the top portion and the bottom portion the single use seal is simultaneously opened.

11. An apparatus for storing one or more electrodes for an automatic external defibrillator comprising:
a container including a top portion, a bottom portion, a cavity for accepting the one or more electrodes, and at least one lateral ridge extending outward from the clam shell container; and
a mechanically advantaged actuator to create separation between the top portion and the bottom portion as the actuator is manipulated and to simultaneously remove the single use seal.

12. The apparatus according to claim 11, further comprising a single use seal disposed between the top portion and the bottom portion.

13. The apparatus according to claim 12, wherein as actuator is manipulated to create separation between the top portion and the bottom portion the single use seal is simultaneously broken.

14. The apparatus according to claim 11, wherein the container comprises a rigid container.

15. The apparatus according to claim 14, wherein the container comprises a semi-rigid container.

16. A method for storing a plurality of electrodes for an automatic external defibrillator comprising:
mounting the plurality of electrodes in a sealed container;
coupling the plurality of electrodes to the automatic external defibrillator; and
monitoring an impedance between the plurality of electrodes when in the sealed container.

17. The method according to claim 16, further comprising using a handle with a mechanical advantage to open the sealed container.

18. The method according to claim 17, further comprising simultaneously breaking a seal while opening the container.

19. The method according to claim 17, further comprising removing a seal from the sealed container while separating a top of the container from a bottom of the container.

20. The method according to claim 16, further comprising monitoring a state of the sealed container, said state including whether the sealed container is open or closed.
